(19) United States
(12) Patent Application Publication
(10) Pub. No.: US 2015/0258229 A1
(43) Pub. Date: Sep. 17, 2015

(54) DISPLAY APPARATUS

(71) Applicant: SAMSUNG DISPLAY CO., LTD., Yongin-City (KR)

(72) Inventors: Youngsang HA, Hwaseong-si (KR); Jongsung BAE, Hwaseong-si (KR); Byongtae RYU, Seoul (KR)

(21) Appl. No.: 14/505,711

(22) Filed: Oct. 3, 2014

(30) Foreign Application Priority Data
Mar. 14, 2014 (KR) 10-2014-0030295

(51) Int. Cl. A61L 2/10; A61L 2/18; A61L 2/20; B08B 1/00

(52) U.S. Cl. CPC A61L 2/10; B08B 1/006; A61L 2/186; A61L 2/202; A61L 2/206; A61L 2/208

(57) ABSTRACT

A display apparatus may include a display wound around a central axis in one direction and configured to be released in another direction opposite to the one direction. The display apparatus may further include a sterilization unit disposed adjacently to the display and configured to sterilize at least one wound surface of the display.

### CROSS-REFERENCE TO RELATED APPLICATION

Korean Patent Application No. 10-2014-0030295, filed on Mar. 14, 2014, in the Korean Intellectual Property Office, and entitled: “Display Apparatus,” is incorporated by reference herein in its entirety.

### BACKGROUND

1. Field

Exemplary embodiments relate to a display apparatus.

2. Description of the Related Art

Display apparatuses may be used to display images in various information processing devices such as a television, a monitor, a laptop personal computer, and a cellular phone. Display apparatuses may include a display for displaying images.

Displays may be liquid crystal displays, organic light emitting displays, electrowetting displays, or electrophoretic displays.

### SUMMARY

A display apparatus may include a display including a front surface and a back surface. The display may be wound in a second direction around a central axis that is extended in a first direction that is perpendicular to the second direction, the display being releasable in a direction opposite to the second direction. The display may further include a sterilization unit adjacent to the display and configured to sterilize a wound surface of the display. The front surface and the back surface may face each other and may be spaced apart from each other.

The display apparatus may further including a housing including a containing space including the wound display, the housing being coupled with the sterilization unit.

The housing may include a wound portion that is wound along the back surface of the display in the second direction in the containing space.

The sterilization unit may include a light source configured to emit ultraviolet light and a light guiding unit coupled with the light source and wound along the front surface of the display in the second direction in the containing space.

The light source may be disposed on the central axis.

The sterilization unit may further include a driver configured to turn the light source on and off at a predetermined maintenance times.

The sterilization unit may include an encapsulation part wound along the front surface of the display in the second direction in the containing space, and a sterilizing agent contained in the encapsulation part.

The sterilization unit may further include a nozzle on a surface of the encapsulation part. The nozzle may face the front surface of the display and may be configured to spray the sterilizing agent.

The sterilization unit may further include a wiper on one side of the housing, the wiper being configured to be in contact with the front surface of the display when the display is released in the direction opposite to the second direction.

The housing may further include a cover that is parallel to a plane of a cross-section that is perpendicular to the central axis, the cover being coupled with the housing.

The sterilization unit may include a light source on one surface of the cover, the light source being configured to emit ultraviolet light onto the display.

The sterilization unit may include a sterilizing agent contained in the cover and a nozzle on one surface of the cover, the nozzle being configured to spray the sterilizing agent onto the display.

The sterilization unit may further include a controller coupled with the cover, the controller being configured to control spraying of the sterilizing agent.

A display apparatus may include a display, a case covering the display, and a sterilization unit coupled with the cover and configured to sterilize at least one surface of the display.

The case may include a first cover covering or exposing a front surface of the display, and a second cover connected to the first cover and covering a back surface of the display.

The sterilization unit may include a light source in one side of the first cover, the light source being configured to emit ultraviolet light.

The first cover may be formed from a light guiding element.

The sterilization unit may include a sterilizing agent in the first cover, and a nozzle on one surface of the first cover, the nozzle being configured to spray the sterilizing agent onto the front surface of the display.

### BRIEF DESCRIPTION OF THE DRAWINGS

Features will become apparent to those of ordinary skill in the art by describing in detail exemplary embodiments with reference to the attached drawings in which:

FIG. 1 illustrates a view of a display apparatus with an unwounded display according to an embodiment;

FIG. 2 illustrates a perspective view of the display apparatus including the display of FIG. 1;

FIG. 3 illustrates a cross-sectional view illustrating the display apparatus taken along a line I-I′ shown in FIG. 2;

FIG. 4 illustrates a perspective view of a display apparatus including a display according to another embodiment;

FIG. 5 illustrates a cross-sectional view of the display apparatus taken along a line II-II′ shown in FIG. 4;

FIGS. 6 and 7 illustrate respectively exploded perspective views illustrating parts of display apparatuses of exemplary embodiments;

FIG. 8 illustrates a perspective view of a display apparatus with an unfolded case according to still another embodiment;

FIG. 9 illustrates a perspective view of the display apparatus of the FIG. 8 with a closed case;

FIG. 10 illustrates a cross-sectional view of the display apparatus taken along a line shown in FIG. 9; and

FIG. 11 illustrates a cross-sectional view of the display apparatus according to an additional embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

It will be further understood that the terms “comprises,” “comprising,” “includes,” and/or “including” used herein specify the presence of stated features or components, but do not preclude the presence or addition of one or more other features or components. It will be understood that when a layer, region, or component is referred to as being “on” or “formed on,” another layer, region, or component, it can be directly or indirectly on or formed on the other layer, region, or component. That is, for example, intervening layers, regions, or components may be present. It will be understood that although the terms “first”, “second”, etc. may be used herein to describe various components, these components should not be limited by these terms. These terms are only used to distinguish one component from another. For example, within the scope of exemplary embodiments, a first component may be designated as a second component, and similarly, the second component may be designated as the first component. Singular expressions, unless defined otherwise in contexts, include plural expressions.

Hereinafter, embodiments will be described in detail with reference to the attached drawings.

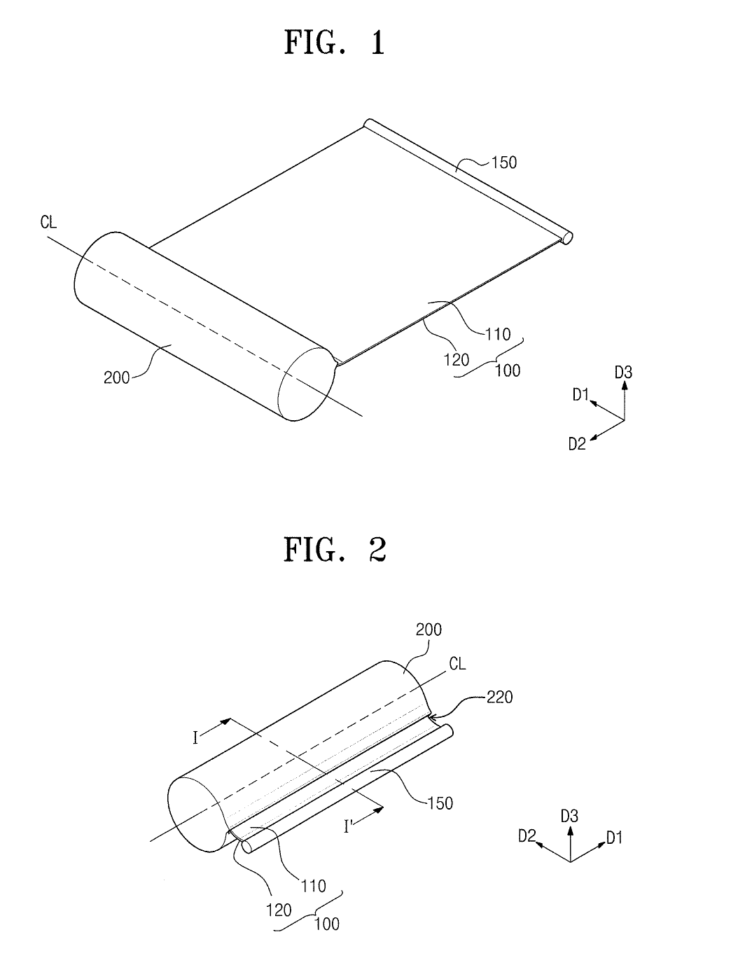

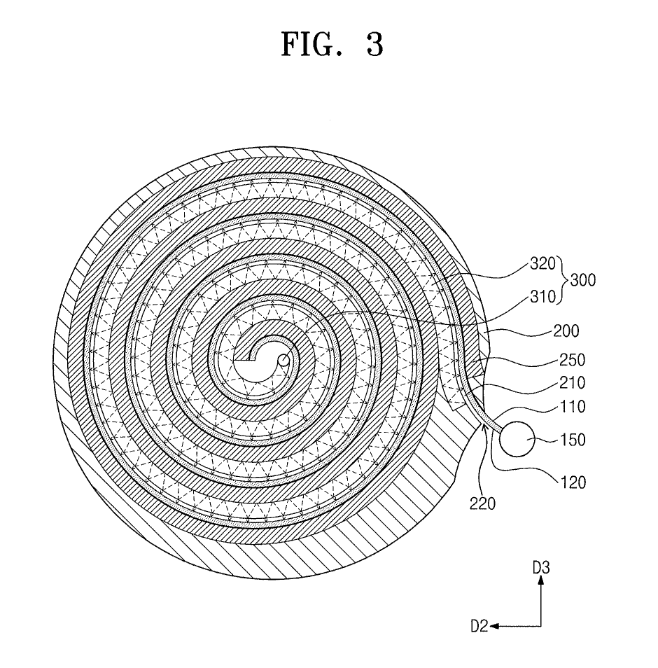

FIG. 1 illustrates a perspective view of a display apparatus with an unwounded display according to an embodiment. FIG. 2 illustrates a perspective view of the display apparatus including the display, and FIG. 3 illustrates a cross-sectional view of the display apparatus taken along a line I-I′ of FIG. 2.

Referring to FIGS. 1 to 3, the display apparatus may include a display 100 configured to display an image, a holder 150 provided on one side of the display 100, a housing 200 including the display 100, and a sterilization unit 300 for sterilizing at least one side of the display 100.

The display 100 may include a front surface 110 and a back surface 120 and may display images using the front surface 110. The display 100 may use a substrate formed of flexible plastic material.

The display 100 having flexibility may be one of an organic emission display and a plasma display. However, not limited thereto, the display 100 having flexibility may be one of a liquid crystal display, an electrowetting display, an electrophoretic display, and a microelectromechanical system (MEMS) display.

The display 100 may be wound around a central axis CL extended in a first direction D1 and wound in a second direction D2 vertical to the first direction D1. And the display 100 may be unwound in a direction opposite to the second direction D2.

The holder 150 may be extended along one side of the display 100 in the first direction D1. The holder 150 may maintain a shape of the display 100 and prevent the display 100 from being wound while displaying images. Also, the holder 150 may be used to allow a user to wind the display 100 or to unwind the wound display 100.

As shown in FIG. 1, the holder 150 may be pulled in the direction opposite to the second direction D2, thereby unwinding the display 100. The front surface 110 of the display 100 may configured to display the image. Also, as shown in FIG. 2, the display may be wound when not being used. In FIG. 2, for convenience of description, a part of the display 100 is shown as being exposed outwards. However, the display 100 may be included in the housing 200 without an exposed part.

Although not shown in the drawing, the display apparatus may include a spring provided on the other side of the display 100 to be located on the central axis CL. Herein, the holder 150 may be instantaneously pulled in the direction opposite to the second direction D2 and then may be released, thereby winding the display 100. The spring may store external force applied to unwind the display 100 as an elastic restoration force and may allow the display 100 to be wound using the stored elastic restoration force without an external power source.

The housing 200 may contain the wound display 100. The housing 200 has a containing space 210 for including the display 100. On one surface of the housing 200, an opening 220 may be formed. The display 100 may be inserted into the containing space 210 or may be withdrawn from the containing space 210 through the opening 220.

The housing 200 may include a wound portion 250 wound along the back surface 120 of the display 100 in the second direction D2 in the containing space 210.

When the display 100 is wound, the wound portion 250 may allow the front surface 110 and the back surface 120 of the display 100 to face each other while being spaced from each other. The wound portion 250 may be formed of various materials such as plastic, metal, and rubber.

The sterilization unit 300 may be disposed adjacently to the display 100 and may sterilize at least one of wound surfaces of the display 100. For convenience of description, in exemplary embodiments, the sterilization unit 300 will be described as sterilizing the wound front surface 110 of the display 100. The sterilization unit 300 may be coupled with the housing 200.

In exemplary embodiments, the sterilization unit 300 may include a light source 310 and a light guiding unit 320.

The light source 310 may be one of a fluorescent lamp, a cold cathode fluorescent lamp (CCFL), and a light emitting diode (LED) lamp. The light source 310 may configured to emit light having a wavelength of from about 250 nm to about 400 nm and may destroy germs on the display 100.

However, the light source 310 is not limited thereto and may be a lamp capable of emitting ultraviolet light.

The light source 310 may be coupled with the light guiding unit 320. The light source 310 may be disposed on the central axis CL and may provide light to one side of the light guiding unit 320.

The light guiding unit 320 may provide the light emitted from the light source 310 to the front surface 110 of the display 100.

The light guiding unit 320 may be wound along the front surface 110 of the display 100 in the second direction D2 in the containing space 210. The light guiding unit 320 may be a flexible film.

The light guiding unit 320 may guide the light emitted from the light source 310 to the other side of the light guiding unit 320. As shown in FIG. 3, the light shown as a dotted line may escape from the inside of the light guiding unit 320 and may arrive at the front surface 110 of the display 100 while moving in the light guiding unit 320 through a plurality of numbers of reflection.

In the embodiment, not shown in the drawing, the light guiding unit 320 may include an empty space therein. The light source may be provided on one side of the empty space. A plurality of microholes, through which the light emitted from the light source 310 may penetrate, may be formed on one surface of the light guiding unit 320 facing the front surface 110 of the display 100. An inner surface of the light guiding unit 320 may be formed by a reflecting element. The reflecting element may reflect the light provided to the empty space toward the microholes. The light passing through the microholes may be provided to the front surface 110 of the display 100.

In the present exemplary embodiment, the sterilization unit 300 may further include a driver (not shown) that may control turning on/off the light source 310 and respective maintenance times of the turning on/off. The driver may control the light source 310 to provide the display 100 with the light for a predetermined time. According thereto, the display 100 may be sterilized for the predetermined time in the predetermined condition. For example, after the display 100 is wound, when an unused time of the display is greater than the predetermined time, the driver may turn on the light source 310.

As described above, the display apparatus includes the sterilization unit 300, and may thereby sterilize the display 100.

Hereinafter, additional embodiments will be described with reference to the drawings. For convenience, duplicative descriptions may be omitted.

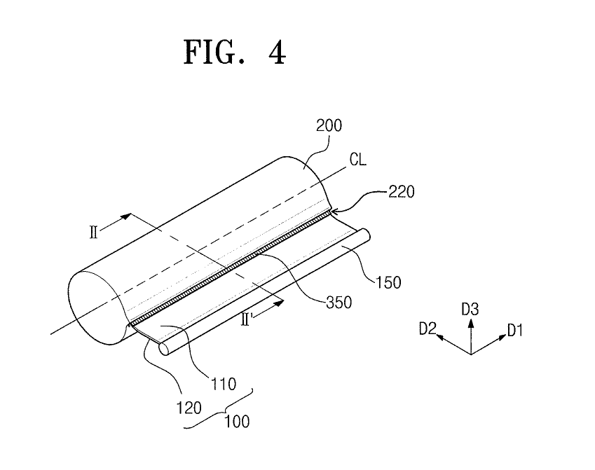

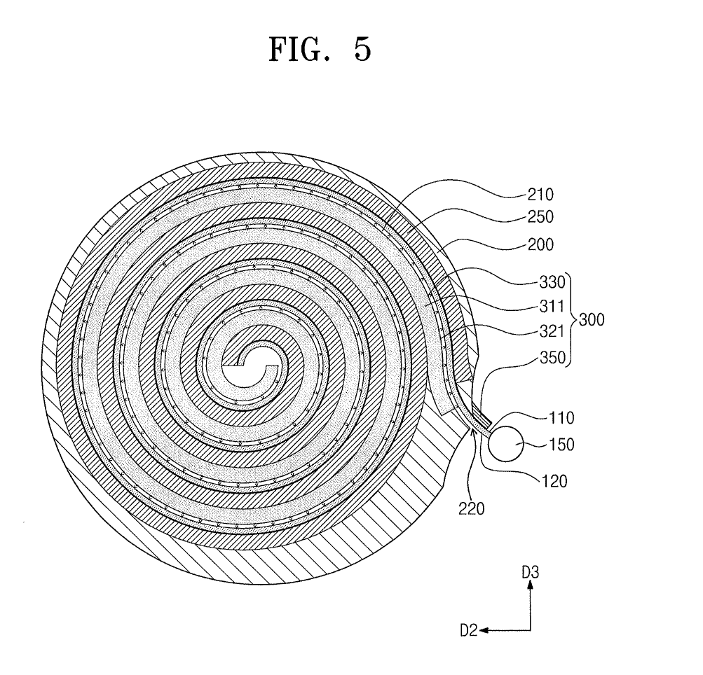

FIG. 4 illustrates a perspective view of a display apparatus including the display according to another embodiment, and FIG. 5 illustrates a cross-sectional view of the display apparatus taken along a line II-II′ of FIG. 4. Hereinafter, referring to FIGS. 1, 4, and 5, additional embodiments will be described.

A sterilization unit 300 may be disposed adjacently to a display 100 and may sterilize at least one of wound surfaces of the display 100.

The sterilization unit 300 may include a sterilizing agent 311, an encapsulation part 321, and a nozzle 330.

The sterilizing agent 311 may be adapted to sterilize the front surface 110 of the display 100 and may be one of gaseous sterilizing agents such as ethylene oxide and hydrogen peroxide, and ozone and liquid sterilizing agents such as hydrogen peroxide, peracetic acid, glutaraldehyde, and o-phthalaldehyde.

The sterilizing agent 311 may be contained in the encapsulation part 321 that will be described later.

The encapsulation part 321 may contain the sterilizing agent 311.

Although not shown in the drawing, the encapsulation part 321 may have an empty space formed therein. The sterilizing agent 311 may be contained in the empty space.

The encapsulation part 321 may be wound along the front surface 110 of the display 100 in the second direction D2 in the containing space 210 of the housing 200.

The nozzle 330 may be disposed on a surface of the encapsulation part 321 opposite to the front surface 110 of the display 100. The nozzle 330 may spray the sterilizing agent 311 to the front surface 110 of the display 100. The sterilization unit 300 may include a plurality of nozzles 330.

The nozzle 330 may pitch and roll within a predetermined angle range, for example, within a range of about 45° to the left side or of about 45° to the right side based on a state in which the nozzle 330 faces the front surface 110 of the display 100. As a result, a spray area of the nozzle 330 may become broader.

The sterilization unit 300 may further include the wiper 350 for removing an excessive amount of the sterilizing agent 311 sprayed onto the display 100.

The wiper 350 may be disposed on one side of the housing 200. The wiper 350 may be in contact with the front surface 110 of the display 100 while the display 100 is released in the direction opposite to the second direction D2, thereby reducing or preventing an outflow of the sterilizing agent 311 sprayed onto the front surface 110 of the display 100.

The wiper 350 may have a substantially identical length to a side of the display 100 extended in the first direction D1.

The wiper 350 may apply slight pressure to the front surface 110 of the display 100. Also, although not shown in the drawing, the sterilization unit 300 may further include a wiper or an elastic element that provides a slight pressure to the back surface 120 of the display 100.

The wiper 350 may be formed of a flexible element capable of being easily bent while providing the pressure to the front surface 110 of the display 100. Also, the wiper 350 may include an absorption element, for example, a sponge or a cloth, capable of absorbing the excessive amount of the sterilizing agent 311.

The wiper 350 may be attached to the housing 200 to be detachable. Accordingly, an exhausted wiper may be removed, and a new wiper may be installed, thereby replacing the wiper 350.

The sterilization unit 300 may further include a spray controller (not shown) for spraying the sterilizing agent 311. The controller may control the sterilizing agent 311 to be sprayed a predetermined number of times while the display 100 is being wound. The controller may be disposed on the one side of the housing 200 and may be, for example, a button (not shown). When the controller is a button, the user may push the button to spray the sterilizing agent 311. Pressure applied to the button may be transferred to the encapsulation part 321, thereby spraying the sterilizing agent 311 through the nozzle 330.

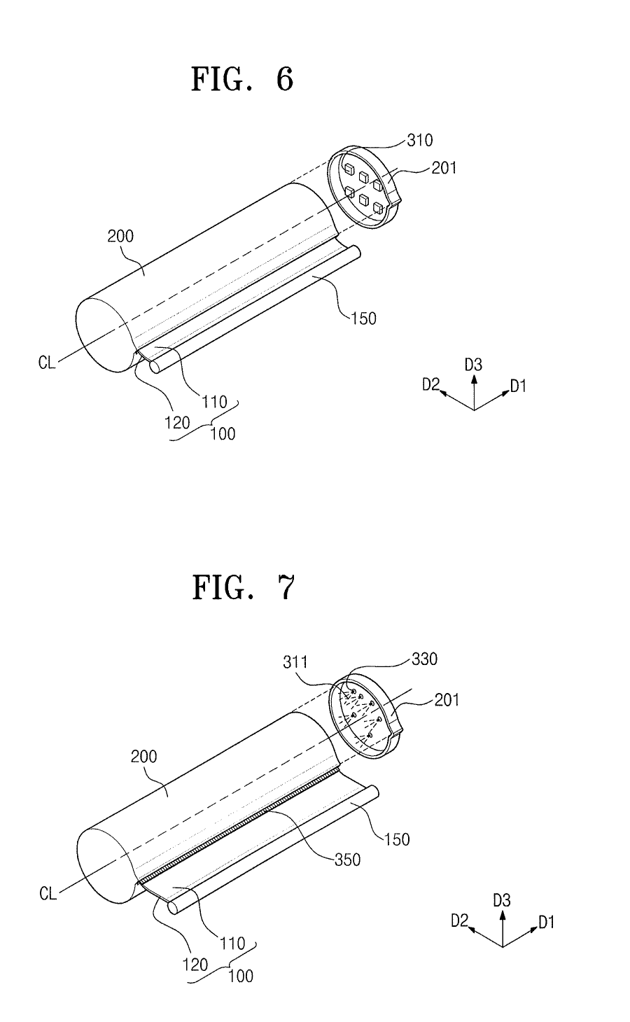

FIG. 6 illustrates an exploded perspective view of a part of the display apparatus.

A housing 200 may contain a wound display 100. The housing 200 may further include a cover 201.

The cover 201 may be parallel to a plane of a cross-section that is perpendicular to a central axis CL and may be coupled with the housing 200.

A sterilization unit 300 may include a light source 310 configured to emit ultraviolet light capable of destroying germs.

The light source 310 may be disposed on one surface of the cover 201 and may provide the light to the wound display 100. There may be one or more light sources 310.

The sterilization unit 300 may further include a driver (not shown) that may control turning on/off the light source 310 and respective maintenance times of the turning on/off. The driver may control the light source 310 to provide the display 100 with the light for a predetermined time. Accordingly, the display 100 may be sterilized for the predetermined time in a predetermined condition. For example, after the display 100 is wound, when an unused time of the display is greater than the predetermined time, the driver may turn on the light source 310.

FIG. 7 illustrates an exploded perspective view of a part of the display apparatus.

A housing 200 may contain a wound display 100. The housing 200 may further include a cover 201. The cover 201 may be parallel to a vertical cross-section of a central axis CL and may be coupled with the housing 200.

The sterilization unit 300 may include a sterilizing agent 311 and a nozzle 330.

The sterilizing agent 311 may be contained inside the cover 201.

Although not shown in the drawing, the cover 201 may include an empty space therein. The sterilizing agent 311 may be contained in the empty space.

The nozzle 330 may be disposed on one surface of the cover 201 and may spray the sterilizing agent 311 to the display 100.

The sterilization unit 300 may further include a wiper 350 for removing the sterilizing agent 311 sprayed onto the display 100.

The wiper 350 may be disposed on one side of the housing 200. The wiper 350 may be in contact with the front surface 110 of the display 100 while the display 100 is released in a direction opposite to a second direction D2, thereby preventing an outflow of the sterilizing agent 311 sprayed onto the front surface 110 of the display 100.

The wiper 350 may have a substantially identical length to a side of the display 100 extended in a first direction D1.

The sterilization unit 300 may further include a spray controller for spraying the sterilizing agent 311. The controller may control the sterilizing agent 311 to be sprayed a predetermined number of times while the display 100 is being wound. The controller may be disposed on the cover 201 and may be a button (not shown). When the controller is a button, a user may push the button, thereby spraying the sterilizing agent 311. Pressure applied to the button may be transferred into the cover 201, thereby resulting in spraying of the sterilizing agent 311 through the nozzle 330.

As described above a display apparatus has been described that may sterilize a wound display; however, embodiments are not limited thereto, and the display apparatus may sterilize a flat panel display.

Hereinafter, additional embodiments will be described with reference to the drawings. For convenience, duplicative descriptions may be omitted.

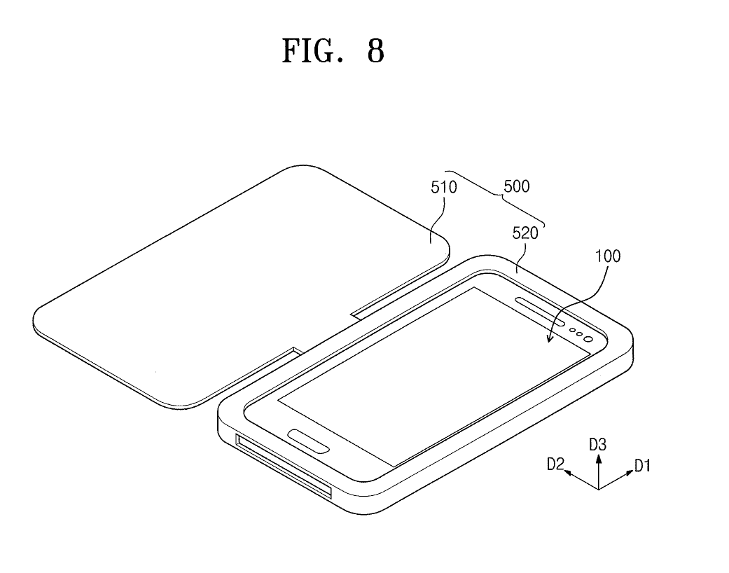

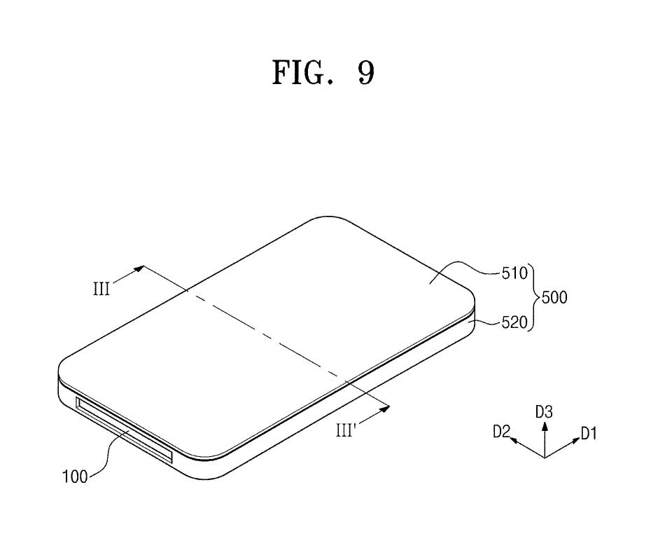

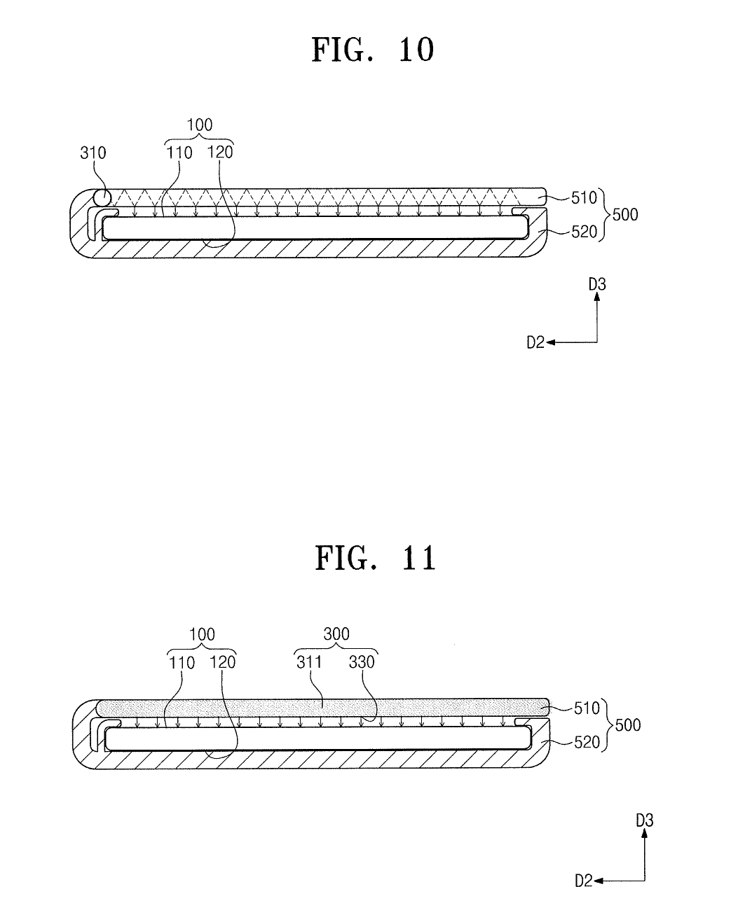

FIG. 8 illustrates a perspective view of a display apparatus with an unfolded case according to still another embodiment, FIG. 9 illustrates a perspective view of the display apparatus with a closed case, and FIG. 10 illustrates a cross-sectional view of the display apparatus taken along a line of FIG. 9.

Referring to FIGS. 8 to 10, a display apparatus may include a display 100 configured to display an image, a case 500 covering the display 100, and a sterilization unit 300 for sterilizing at least one surface of the display 100.

The display 100 may include front surface 110 and a back surface 120. The front surface 110 of the display 100 may configured to display an image.

The display 100 may be one of a liquid crystal display and an organic light emitting display. However, the display 100 is not limited thereto and may be one of a plasma display, an electrowetting display, an electrophoretic display, and a microelectromechanical system (MEMS) display.

The case 500 may include a first cover 510 covering or exposing the front surface 110 of the display 100 and a second cover 520 connected to the first cover 510 and covering the back surface 120 of the display 100. In exemplary embodiments, the second cover 520 may cover not only the back surface 120 of the display 100 but also a side of the display 100 and a part of an edge of the front surface 110 of the display 100.

The first cover 510 may be formed of a light guiding element.

The sterilization unit 300 may include a light source 310 configured to emit ultraviolet light capable of destroying germs.

The light source 310 may be disposed on one side of the first cover 510. The light source 310 may be extended along one side of the display 100 in a first direction D1. However, according to a size of the display 100, a length and a position of the light source 310 may vary. Also, the sterilization unit may include a plurality of light sources 310. The plurality of light sources 310 may be disposed in the first cover 510 while being spaced from one another.

The first cover 510 may guide the light emitted from the light source 310 to the other side of the first cover 510. The light shown as a dotted line in FIG. 10 may escape from the first cover 510 and may illuminate the front surface 110 of the display 100 while moving in the first cover 510 through multiple internal reflections.

Although not shown in the drawings, the first cover 510 may include an empty space therein. The light source 310 may be provided on one side of the empty space. A plurality of microholes, through which the light emitted from the light source 310 may penetrate, may be formed on one surface facing the front surface 110 of the display 100. An inner surface of the first cover 510 may be formed of a reflecting element. The reflecting element may reflect the light provided to the empty space toward the microholes. The light passing through the microholes may be provided to the front surface 110 of the display 100.

The second cover 520 may be formed of material such as plastic or a metal, thereby protecting the display 100 from external impacts. However, the second cover 520 may also be formed of the light guiding element (not shown). When the second cover 520 is formed of the light guiding element, the second cover 520 may receive the light emitted from the light source 310 and may transfer the light in the second cover 520. The light may escape from the second cover 520 and may illuminate the back surface 120 of the display 100 while moving in the second cover 520 through multiple internal reflections.

The sterilization unit 300 may further include a driver (not shown) controlling turning on/off the light source 310 and respective maintenance times of the turning on/off. The driver may control the light source 310 to provide the display 100 with the light for a predetermined time. According thereto, the display 100 may be sterilized for the predetermined time in a predetermined condition.

FIG. 11 illustrates a cross-sectional view of a display apparatus according to another embodiment. FIG. 11 illustrates the display apparatus taken along a line shown in FIG. 9.

Referring to FIGS. 8, 9, and 11, a display apparatus may include a display 100 configured to display images, a case 500 covering the display 100, and a sterilization unit 300 for sterilizing at least one surface of the display 100.

The case 500 may include a first cover 510 covering or exposing the front surface 110 of the display 100 and a second cover 520 connected to the first cover 510 and covering the back surface 120 of the display 100. In exemplary embodiments, the second cover 520 may cover not only the back surface 120 of the display 100 but also a side of the display 100 and a part of an edge of the front surface 110 of the display 100.

The sterilization unit 300 may include a sterilizing agent 311 and a nozzle 330.

The sterilizing agent 311 may be contained inside the first cover 510. Although not shown in the drawing, the first cover 510 may include an empty space therein. The sterilizing agent 311 may be contained in the empty space.

The nozzle 330 may be disposed on a surface of the first cover 510, opposite to the front surface 110 of the display 100. The nozzle 330 may spray the sterilizing agent 311 from the first cover 510 to the front surface 110 of the display 100. The sterilization unit may include a plurality of nozzles 330.

The sterilization unit 300 may further include a spray controller for spraying the sterilizing agent 311. The controller may be disposed on the first cover 510 and for example, may be a button. When the controller is a button, a user may push the button to spray the sterilizing agent 311. Pressure applied to the button may be transferred into the first cover 510, thereby spraying the sterilizing agent 311 through the nozzle 330.

According to exemplary embodiments, a display apparatus may include a sterilized display. Accordingly, a user may be provided with a clean display apparatus.

By way of summation and review, displays may be in frequent contact with the hands of users and may become polluted with germs on surfaces of the displays. Exemplary embodiments herein provide a display apparatus having a sterilization function that may reduce germs or prevent the accumulation of germs on surfaces of the display. In particular, exemplary embodiments include a display apparatus including a display configured to display images and a sterilization unit configured to sterilize the display.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A display apparatus comprising:
a display including a front surface and a back surface,
wherein the display is wound around a central axis extending in a first direction such that winding of the display is in a second direction normal to the first direction, the second direction being a winding direction,
a sterilization unit adjacent to the display to sterilize a wound surface of the display,
wherein the front surface and the back surface face each other and are spaced apart from each other in a wound condition of the display.

2. The display apparatus as claimed in claim 1, further including a housing including a containing space including the display in the wound condition, the housing being coupled with the sterilization unit.

3. The display apparatus as claimed in claim 2, wherein the housing includes a wound portion that is wound along the back surface of the display in the second direction in the containing space.

4. The display apparatus as claimed in claim 3, wherein the sterilization unit includes:
a light source configured to emit ultraviolet light; and
a light guiding unit coupled with the light source and wound along the front surface of the display in the second direction in the containing space.

5. The display apparatus as claimed in claim 4, wherein the light source is disposed on the central axis.

6. The display apparatus as claimed in claim 5, wherein the sterilization unit further includes a driver configured to turn the light source on and off at a predetermined maintenance times.

7. The display apparatus as claimed in claim 3, wherein the sterilization unit includes:
an encapsulation part wound along the front surface of the display in the second direction in the containing space; and
a sterilizing agent contained in the encapsulation part.

8. The display apparatus as claimed in claim 7, wherein the sterilization unit further includes a nozzle on a surface of the encapsulation part,
wherein the nozzle faces the front surface of the display and is configured to spray the sterilizing agent.

9. The display apparatus as claimed in claim 8, wherein the sterilization unit further includes a wiper on one side of the housing, the wiper being configured to be in contact with the front surface of the display when the display is unwound and released in a direction opposite to the second winding direction.

10. The display apparatus as claimed in claim 3, wherein the housing further includes a cover that is parallel to a plane of a cross-section that is perpendicular to the central axis, the cover being coupled with the housing.

11. The display apparatus as claimed in claim 10, wherein the sterilization unit includes a light source on one surface of the cover, the light source being configured to emit ultraviolet light onto the display.

12. The display apparatus as claimed in claim 10, wherein the sterilization unit includes:
a sterilizing agent contained in the cover; and
a nozzle on one surface of the cover, the nozzle being configured to spray the sterilizing agent onto the display.

13. The display apparatus as claimed in claim 12, wherein the sterilization unit further includes a wiper on one side of the housing, the wiper being configured to contact the front surface of the display when the display is released in a direction opposite to the second winding direction.

14. The display apparatus as claimed in claim 13, wherein the sterilization unit further includes a controller coupled with the cover, the controller being configured to control spraying of the sterilizing agent.

15. A display apparatus comprising:
a display;
a case covering the display; and
a sterilization unit coupled with the cover to sterilize at least one surface of the display.

16. The display apparatus as claimed in claim 15, wherein the case includes:
a first cover covering or exposing a front surface of the display; and
a second cover connected to the first cover and covering a back surface of the display.

17. The display apparatus as claimed in claim 16, wherein the sterilization unit includes a light source in one side of the first cover, the light source being configured to emit ultraviolet light.

18. The display apparatus as claimed in claim 17, wherein the first cover is formed from a light guiding element.

19. The display apparatus as claimed in claim 18, wherein the sterilization unit further includes a driver activatable to turn the light source on and off at a predetermined maintenance times.

20. The display apparatus as claimed in claim 16, wherein the sterilization unit includes:
a sterilizing agent in the first cover; and
a nozzle on one surface of the first cover to spray the sterilizing agent onto the front surface of the display.
